(19) United States
(12) Patent Application Publication
(10) Pub. No.: US 2025/0002584 A1
(43) Pub. Date: Jan. 2, 2025

(54) ANTI-B7-H6 ANTIBODY, FUSION PROTEINS, AND METHODS OF USING THE SAME

(71) Applicant: THE TRUSTEES OF DARTMOUTH COLLEGE, Hanover, NH (US)

(72) Inventors: Charles L. SENTMAN, Grantham, NH (US); Tong ZHANG, Beijing (CN)

(21) Appl. No.: 18/767,080

(22) Filed: Jul. 9, 2024

Related U.S. Application Data
(62) Division of application No. 17/146,814, filed on Jan. 12, 2021, now Pat. No. 12,065,492.
Application No. 17/146,814 is a division of application No. 15/784,342, filed on Oct. 16, 2017, now Pat. No. 11,034,766.
Application No. 15/784,342 is a division of application No. 14/399,835, filed on Nov. 7, 2014, now Pat. No. 9,790,278.
(60) Provisional application No. 61/643,456, filed on May 7, 2012.
(60) Provisional application No. 61/705,227, filed on Sep. 25, 2012.

(51) Int. Cl. C07K 16/28; A61K 39/395; C07K 14/705; C07K 14/725

(52) U.S. Cl. CPC C07K 16/2827; A61K 39/3955; C07K 14/7051; C07K 14/70521; C07K 16/2809; C07K 2317/24; C07K 2317/31; C07K 2317/622; C07K 2319/00; C07K 2319/33

(57) ABSTRACT

Antibodies, chimeric antigen receptors, and bispecific T-cell engagers having specificity for B7-H6 and methods for using the same in the diagnosis and treatment of disorders associated with B7-H6 expression are provided.

### CROSS REFERENCE TO RELATED APPLICATIONS

This application is a divisional of U.S. patent application Ser. No. 17/146,814 filed Jan. 12, 2021, now U.S. Pat. No. 12,065,492, which is a divisional of U.S. patent application Ser. No. 15/784,342, filed Oct. 16, 2017, now U.S. Pat. No. 11,034,766, which is a divisional of U.S. patent application Ser. No. 14/399,835, filed Nov. 7, 2014, now U.S. Pat. No. 9,790,278, which is a U.S. Nat'l Phase of Int'l Appl. No. PCT/US2013/039812, filed May 7, 2013, which claims priority to U.S. Provisional Appl. Nos. 61/705,227, filed Sep. 25, 2012, and 61/643, 456, filed May 7, 2012, the disclosures of each of which are hereby incorporated by reference in their entireties.

### SEQUENCE LISTING DISCLOSURE

The contents of the electronic sequence listing (1148307o001004.xml; Size: 27,988 bytes; and Date of Creation: Jul. 9, 2024) is herein incorporated by reference in its entirety.

This invention was made with government support under contract number CA130911 awarded by the National Institutes of Health. The government has certain rights in the invention.

### BACKGROUND OF THE INVENTION

Natural killer (NK) cells are lymphocytes of the innate immune system that participate in the elimination of tumor cells. In humans, the activating natural cytotoxicity receptors (NCRs) NKp30, NKp44, and NKp46 play a major role in NK cell-mediated tumor cell lysis. NKp30 recognizes B7-H6, a member of the B7 family. Like all known B7 family members, B7-H6 includes two Ig domains with adjacent phase 1 introns in the extracellular region. Importantly, B7-H6 is not detected in normal human tissues but is selectively expressed on a variety of human tumor cell lines, including T and B lymphomas, melanomas, and carcinomas (Brandt, et al. (2009) J. Exp. Med. 206:1495-1503). Furthermore, B7-H6 expression on tumor cells triggers NKp30-specific NK cell cytotoxicity and cytokine secretion. Thus, B7-H6 functions as a tumor-induced self-molecule that alerts innate immunity to cellular transformation via its interaction with the activating receptor NKp30 (Brandt, et al. (2009) supra).

### SUMMARY OF THE INVENTION

This invention is an isolated antibody, or antigen binding fragment of the antibody, which specifically binds to B7 homolog 6 (B7-H6), wherein the CDR1, CDR2, and CDR3 sequences of the heavy chain variable region are set forth in SEQ ID NO: 5, SEQ ID NO: 6, and SEQ ID NO: 7, respectively; and the CDR1, CDR2, and CDR3 sequences of the light chain variable region are set forth in SEQ ID NO: 8, SEQ ID NO: 9, and SEQ ID NO: 10, respectively. In one embodiment, the antibody has a heavy chain variable domain of SEQ ID NO: 3. In another embodiment, the antibody has a light chain variable domain of SEQ ID NO: 4. In other embodiments, the antibody is humanized. In yet further embodiments, the antibody is conjugated to a synthetic molecule such as a label, cytotoxic agent or therapeutic radioisotope. In still other embodiments, the antibody is a chimeric antigen receptor and the synthetic molecule includes a transmembrane region, an intracellular T-cell receptor signaling domain, e.g., obtained from CD3 zeta, and an optional intracellular domain of a costimulatory protein receptor. In other embodiments, the antibody is a bi-specific T-cell engager and the synthetic molecule includes an antigen binding domain that binds to a T-cell antigen, e.g., CD3. A pharmaceutical composition including the antibody or antigen binding fragment of the invention and a pharmaceutically acceptable carrier is provided, as is a kit containing the antibody or antigen binding fragment, e.g., conjugated to a label.

The invention is also a chimeric antigen receptor, which includes an antigen binding fragment of an antibody that specifically binds to B7 homolog 6, a transmembrane region, and an intracellular T-cell receptor signaling domain. In some embodiments, the transmembrane region and intracellular T-cell receptor signaling domain are obtained from CD3 zeta. In other embodiments, the chimeric antigen receptor further includes an intracellular signaling domain of a costimulatory protein receptor.

The invention further provides a bi-specific T-cell engager including an antigen binding fragment of an antibody that specifically binds to B7 homolog 6, and an antigen binding domain that binds to a T-cell antigen, wherein in some embodiments the T-cell antigen is CD3.

Methods for killing or inhibiting the growth of cells expressing B7 homolog 6 and treating a disease or condition associated with aberrant expression of B7 homolog 6 are also provided using the antibodies described herein.

### BRIEF DESCRIPTION OF THE DRAWINGS

FIG. 1 shows the structure of an anti-B7H6 CAR.

FIGS. 2A-2B show that NKp30 Chimeric Antigen Receptor (CAR) and anti-B7H6 CAR-modified T cells respond to NKp30 ligand positive cells by producing IFN-γ. T-cells expressing wild type B7-H6 (WT), anti-B7-H6 CAR (aB7H6-28-z), or a NKp30-CD28-CD3 zeta fusion protein (30-28-z) were contacted with media, RMA cells, RMA cells that express B7-H6 or K562 cells (10⁵ cells) (FIG. 2A), or with peripheral blood mononuclear cells (PBMCs, 10⁵ cells) or immature DCs (iDCs, 2×10⁴ cells) (FIG. 2B) for 24 hours and the level of IFN gamma production was measured with ELISA. Results are shown in mean±SD.

FIG. 3 shows the expression of HLA-DR, CD83, CD86, NKp30 ligands, and B7-H6 on immature dendritic cells (iDC) and mature dendritic cells (mDC).

FIG. 4 shows the expression of B7H6 (using anti-B7H6 monoclonal antibodies) and NKp30 ligands (using soluble NKp30-Ig) on various human cell lines and mouse RMA and RMA-B7H6 cells compared to isotype control staining.

FIGS. 5A-5C show that anti-B7H6 CAR modified T cell increase survival of RMA/B7H6 lymphoma-bearing mice after in vivo treatment. B6 mice were inoculated with RMA/B7H6 (10⁵ cells, i.v., day 0). Mock-transduced or anti-B7H6 CAR-modified T cells were administered (5×10⁶, i.v., day 5, 7, 9 FIG. 5A)). Data were presented in Kaplan-Meier survival curves. Data shown were pools of two independent experiments (FIG. 5B). Naïve and surviving mice (from FIG. 5B) were rechallenged with 10⁴ RMA subcutaneously (no B7H6 expression), and tumor area was measured every other day (FIG. 5C). These data show that the surviving mice after CAR therapy were resistant to the same tumor, which indicates induction of an immune response against other tumor antigens because the RMA cells do not express B7H6.

FIGS. 6A-6B show the structure and activity of an anti-B7H6 BiTE. FIG. 6A depicts the fusion between the scFv of an anti-B7H6 antibody and the scFv of an anti-CD3 antibody (OKT3). VH, variable region heavy chain; VL, variable region light chain; and L, linker. FIG. 6B shows that conditioned media containing this BiTE triggers robust killing of B7H6+ tumor cells by human T cells against tumor cells that express B7H6 (RMA-B7H6, K562) but not against tumor cells that do not (RMA). OKT3-activated PBMCs were co-cultured with tumor cell lines at a E:T ratio (Effector Cell to Target Cell ratio) of 5:1 in a 1 to 4 diluted conditioned media containing the anti-B7H6 BiTE or control media. Five hours after co-culturing, cell supernatants were harvested and cytotoxicity was determined by lactate dehydrogenase (LDH) release assay.

FIGS. 7A-7C provides data, which shows that anti-B7H6 BiTE proteins trigger IFN-γ secretion. FIG. 7A, OKT3-activated human T cells were co-cultured with tumor cells at E:T=1:1 ratio (10⁵:10⁵) under various concentrations of huBiTEl. Supernatants were collected after 24 hours and IFN-γ concentrations were determined by ELISA. FIG. 7B, OKT3 -activated T cells were cultured with tumor cells at E:T ratios of 4:1, 1:1, or 0.25:1. HuBiTEl was added to the co-culture at a concentration of 250 ng/mL. Culture supernatants were collected after 24 hours. IFN-γ concentrations were measured by ELISA. FIG. 7C shows that conditioned media containing anti-B7H6 BiTE that an activates mouse T cells triggers robust IFN-γ from mouse T cells when co-cultured with mouse tumor cells expressing B7H6. ConA-activated mouse splenocytes were co-cultured with tumor cell lines at a E:T ratio 5:1 (for B16F10 and B16F10/B7H6, 10⁵:2×10⁴) or 1:1 (for K562, 10⁵:10⁵) in a 1 to 4 diluted conditioned media from 293F cells transfected with mouse Bitel plasmid or control supernatant. After co-culturing (24 hours), supernatants were collected and IFN-γ concentrations were determined by ELISA.

FIGS. 8A-8B show that mouse BiTE1 (anti-B7H6 BiTE) specifically activated mouse T cells to kill B7H6+ tumor cells. ConA-activated mouse splenocytes were co-cultured with a RMA (pre-labeled with 0.1 μM CSFE) and RMA-B7H6 (pre-labeled with 1 μM CSFE) mixture (50% RMA+50% RMA-B7H6; FIG. 8A) or B16F10 and B16F10-B7H6 mixture (50% B16F10+50% B16F10-B7H6; FIG. 8B) at E:T ratio 1:1, 5:1, and 10:1. Eight hours and 24 hours later, cells were harvested and the identity of live cells was determined by flow cytometry. Specific lysis of the B7H6+ cells was calculated based on the control group normalized ratio of RMA:RMA-B7H6 or B16F10:B16F10-B7H6 ratio of each experimental group.

### DETAILED DESCRIPTION OF THE INVENTION

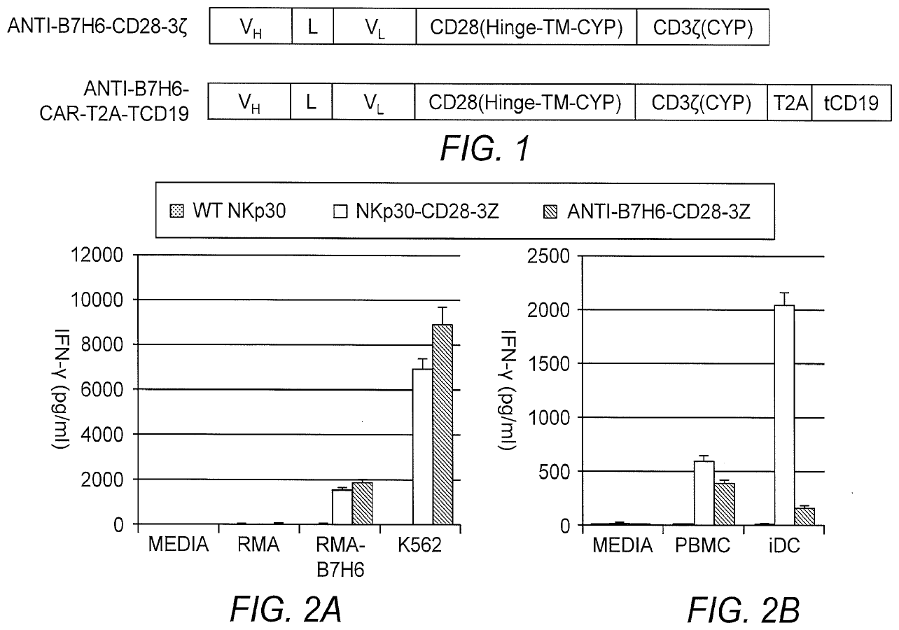

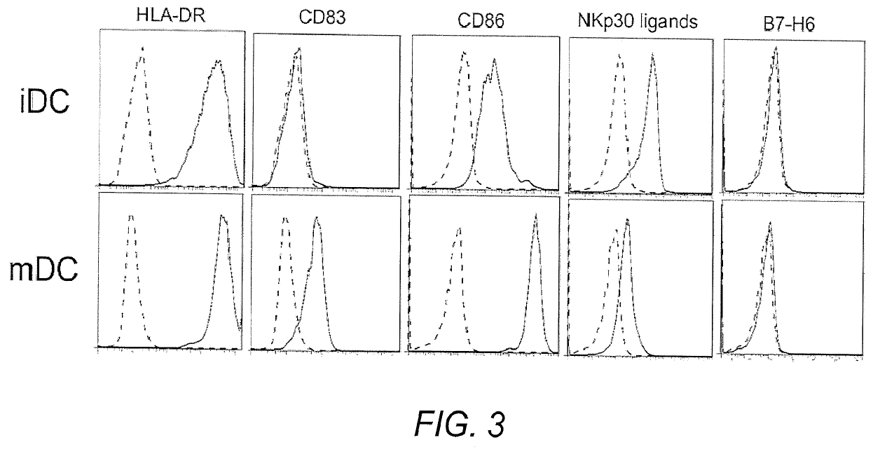

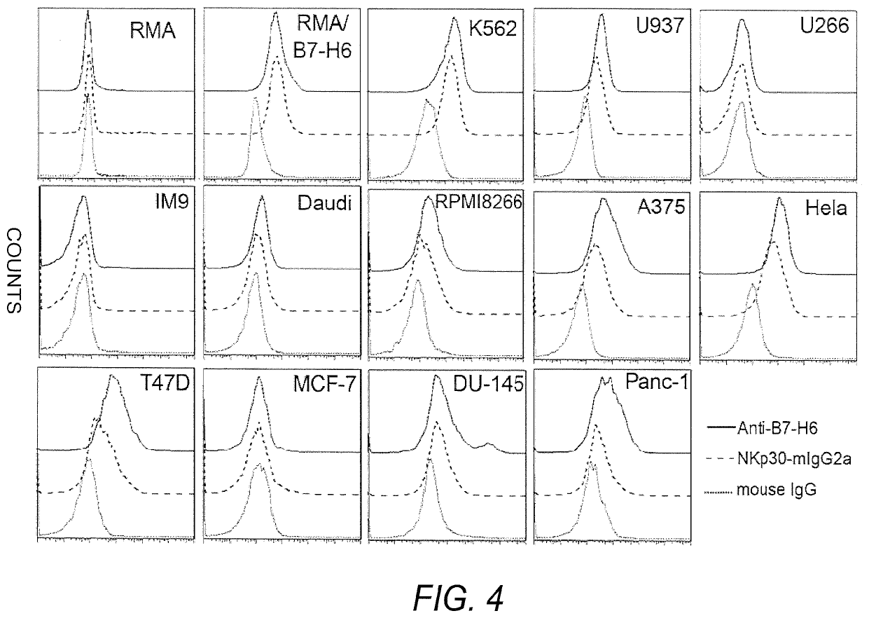

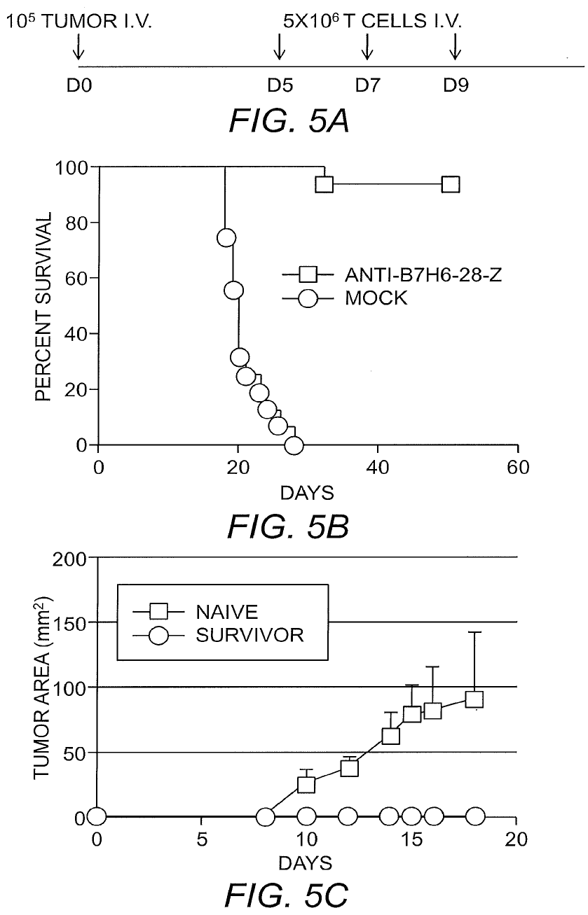

To target B7-H6 in the treatment of cancer, two mouse-derived monoclonal antibodies (mAb) that specifically recognize B7-H6 were generated. Both clones were of the mouse IgG2a subclass. The genes encoding the heavy and light chains of mAb clone 47.39 were isolated. The nucleotide and deduced amino acid were determined. Using these sequences, a chimeric antigen receptor (CAR) was generated, which included the variable portions of heavy (VH) and light (VL) chains of mAb 47.39. Specifically, the anti-B7-H6 CAR included a single chain variable fragment (scFv) which recognized B7-H6, followed by a portion of CD28 molecule (including the hinge, transmembrane and cytoplasmic domains) and the cytoplasmic region of CD3zeta (FIG. 1). Using flow cytometry, it was confirmed that this CAR could recognize the B7-H6 molecule. Furthermore, it was found that T-cells expressing the anti-B7-H6 CAR produced high levels of IFN gamma in response to stimulation by B7-H6-positive cells, i.e., K562 myelogenous leukemia cells and RMA cells that expresses B7-H6 (FIG. 2A), whereas autologous PBMCs and iDCs did not respond to anti-B7-H6 CAR (FIG. 2B). Flow cytometry data showed that dendritic cells (DC) do not express B7-H6 on the cell surface (FIG. 3), whereas cancer cell lines such as K562, U937, A375, Hela, T47D, and Panc-1 do express B7-H6 (FIG. 4) and said expression is correlated with NKp30 expression. In this respect, unlike a NKp30-based CAR, the anti-B7H6 CAR exhibited no reactivity against autologous dendritic cells. Furthermore, anti-B7H6 CAR was shown to redirect T cells to specifically lyse B7H6-positive tumor cells but not B7H6-negative tumor cells. Adoptive transfer of murine T cells expressing the B7H6-specific CAR resulted in improved survival in C57BL/6 mice that had been infused five days earlier with B7H6+ lymphoma cells (FIGS. 5A and 5B). Moreover, mice that survived the first challenge with B7H6⁺ lymphoma cells demonstrated immunity upon rechallenge with B7H6⁺ lymphoma cells (FIG. 5C). Thus, this demonstrates the use of a B7H6-specific CAR for adoptive T cell immunotherapy against B7H6+ tumors.

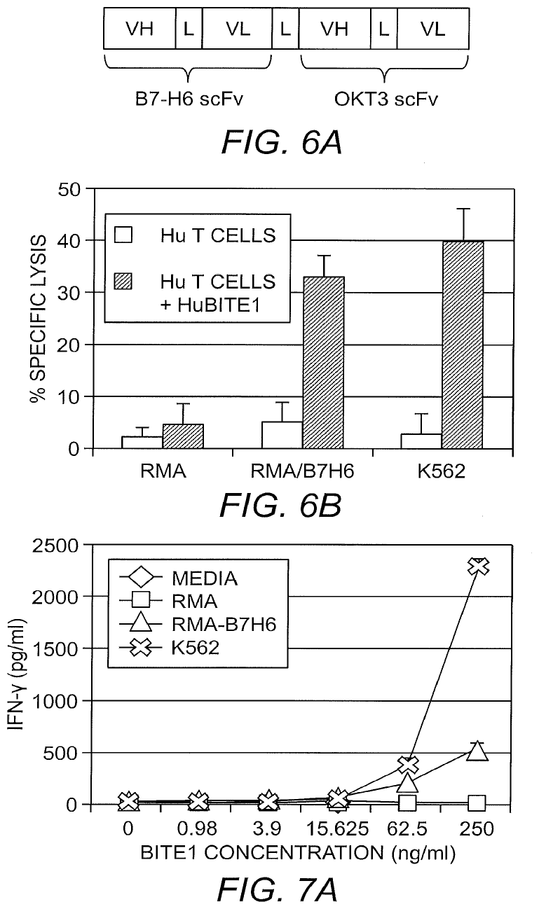

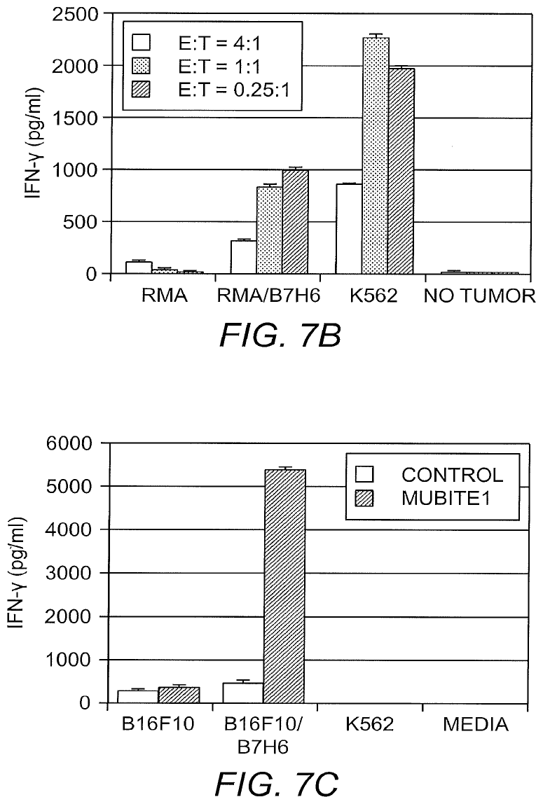

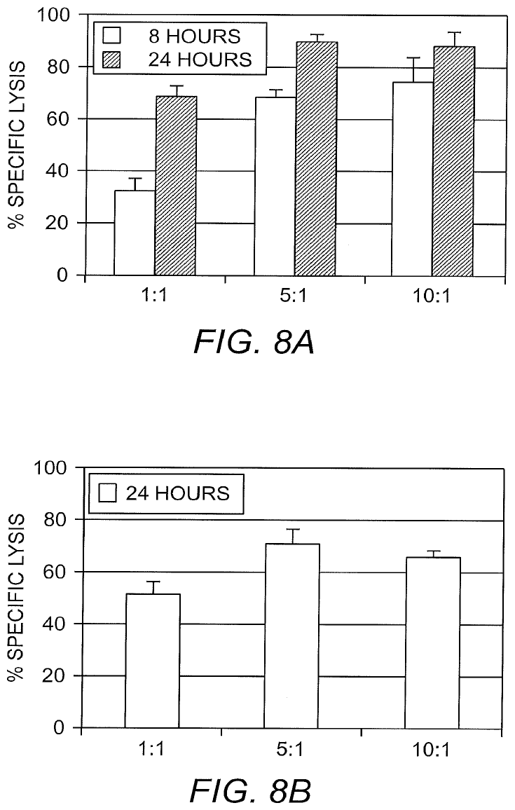

In addition to an anti-B7H6 CAR, an anti-B7H6/anti-CD3 BiTE (bi-specific T-cell engager) was generated (FIG. 6A). Human and mouse versions of the anti-B7H6 BiTE (i.e., huBiTEl and muBiTEl, respectively) were produced and both were capable of specifically lysing B7H6+ tumors (FIGS. 6B, 8A and 8B). Moreover, both the human and mouse versions of the anti-B7H6 BiTE triggered IFN-γ secretion in a T cell and tumor cell co-culture (FIGS. 7A-7C).

Therefore, this invention provides an antibody having specificity for B7-H6, wherein the antibody has a heavy chain encoded by the nucleotide sequence of SEQ ID NO: 1 and a light chain encoded by the nucleotide sequence of SEQ ID NO: 2. Moreover, the invention provides an antibody having specificity for B7-H6, wherein the antibody has a heavy chain amino acid sequence of SEQ ID NO: 3 and a light chain amino acid sequence of SEQ ID NO: 4. More particularly, the invention provides an antibody that specifically binds B7-H6, wherein the heavy chain variable region of the antibody has a CDR1 sequence of Gly-Tyr-Thr-Phe-Thr-Gly-Tyr-Trp (SEQ ID NO: 5), CDR2 sequence of Ile-Leu-Pro-Gly-Thr-Gly-Ser-Thr (SEQ ID NO: 6), and CDR3 sequence of Ala-Ile-Pro-Gly-Pro-Met-Asp-Tyr (SEQ ID NO: 7); and the light chain variable region of the antibody has a CDR1 sequence of Gin-Asp-Ile-Asn-Ser-Tyr (SEQ ID NO: 88, CDR2 sequence of Arg-Ala-Asn (SEQ ID NO: 9) and CDR3 sequence of Leu-Gln-Tyr-Asp-Glu-Phe-Pro-Tyr-Thr (SEQ ID NO: 10). In some embodiments, the antibody can include a heavy chain of SEQ ID NO: 3 in combination with any suitable light chain, such as those described in US 2004/0152105, PCT/US2000/030039 and PCT/IB2010/003411. Likewise, the antibody can include a light chain of SEQ ID NO: 4 in combination with any suitable heavy chain, such as those described in US 2004/0152105, PCT/US2000/030039 and PCT/IB2010/003411.

The antibody can be an isolated antibody having specificity for human B7-H6 and can be a full length antibody or an antibody fragment. The antibody can be polyclonal, monoclonal, recombinant, chimeric, or humanized. Furthermore, the antibody can be of any isotype including without limitation IgA, IgD, IgE, IgG, or IgM. Thus, for example, the antibody can be any IgA such as IgA1 or IgA2,or any IgG such as IgG1, IgG2, IgG3, IgG4, or synthetic IgG. The antibody can also be any antibody fragment having specificity for B7-H6, such as F(ab)₂, Fv, scFv, F(ab′)₂, F(ab), VL, VH, dsFv, Fv, scFv-Fc, (scFv)₂, a diabody, and a bivalent antibody. The antibody can be any modified or synthetic antibody, including, but not limited to, non-depleting IgG antibodies, T-bodies, or other Fc or Fab variants of antibodies.

In addition to a heavy chain of SEQ ID NO: 3, the antibody of the invention can further include a light chain selected from a Fab library using sequential naïve chain shuffling. Likewise, in addition to a light chain of SEQ ID NO: 4, the antibody of the invention can further include a heavy chain selected from a Fab library using sequential naive chain shuffling.

In some embodiments, the invention provides an antibody with avidity for B7-H6 of about 10 μM or less, 5 μM or less, 2 μM or less, 1 μM or less, 500 nM or less, 400 nM or less, 300 nM or less, or 200 nM or less. The invention also provides an antibody with avidity for B7-H6 of about 100 nM or less, about 75 nM or less, about 50 nM or less, about 25 nM or less, about 10 nM or less, or about 5 nM or less. Avidity can be measured using art-known techniques, such as ELISA or BIACORE.

The antibody of the invention can be produced by any suitable technique, for example, using any suitable eukaryotic or non-eukaryotic expression system. In certain embodiments, the antibody is produced using a mammalian expression system. In some embodiments, the heavy chain can be encoded by a DNA sequence such as SEQ ID NO: 1, while the light chain can be encoded by a DNA sequence such as SEQ ID NO: 2.

The antibody of the invention can be produced using a suitable non-eukaryotic expression system such as a bacterial expression system. Bacterial expression systems can be used to produce fragments such as a F(ab)₂, Fv, scFv, F(ab′)₂, F(ab), VL, VH, dsFv, Fv, scFv-Fc, (scFv)₂, and diabodies. Techniques for altering DNA coding sequences to produce such fragments are known in the art.

The antibody of the invention can be conjugated to a synthetic molecule using any type of suitable conjugation. Recombinant engineering and incorporated selenocysteine (e.g., as described in WO 2008/122039) can be used to conjugate a synthetic molecule. Other methods of conjugation can include covalent coupling to native or engineered lysine side-chain amines or cysteine side-chain thiols. See, e.g., Wu, et al. (2005) Nat. Biotechnol. 23:1137-1146. The synthetic molecule can be any molecule such as one targeting a tumor. Of course, it will be understood that the synthetic molecule also can be a protein or an antibody, wherein the resulting fusion protein can be produced by conventional recombinant protein expression systems and methods.

In this respect, particular embodiments include chimeric antigen receptors (CARs). CARs, also known as artificial T cell receptors, chimeric T cell receptors, or chimeric immunoreceptors, are engineered receptors, which graft an arbitrary specificity onto an immune effector cell. Typically, these receptors are used to graft the specificity of a monoclonal antibody onto a T cell, e.g., via retroviral vector expression. The most common form of these molecules are fusions of scFv derived from monoclonal antibodies, fused to CD3-zeta transmembrane and endodomain, i.e., an intracellular T-cell receptor (TCR) signaling domain. Such molecules result in the transmission of a zeta signal in response to recognition by the scFv of its target. “First-generation” CARs typically have the intracellular domain from the CD3 zeta-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell (see FIG. 1A). Preclinical studies have indicated that the second generation of CAR designs improves the antitumor activity of T cells (Maher, et al. (2002) Nat. Biotechnol. 20:70-75; Kowolik, et al. (2006) Cancer Res. 66:10995-11004). More recent, “third-generation” CARs combine multiple signaling domains, such as CD3z-CD28-41BB or CD3z-CD28-OX40, to further augment potency (Zhao, et al. (2009) J. Immunol. 183:5563-5574; Pule, et al. (2005) Mol. Ther. 12:933-941; Zhong, et al. (2010) Mol. Ther. 18:413-420). Accordingly, in one embodiment of this invention, the anti-B7-H6 scFv fragment is included in a CAR.

CARs of this invention can be prepared using standard recombinant protein techniques using sequences of CD3-zeta and other costimulatory molecules known in the art. For example, the human CD3-zeta sequence is available under GENBANK accession number NP_932170, the human CD28 sequence is available under GENBANK accession number NP_006130, the human OX40 sequence is available under GENBANK accession number NP_003318, and the human CD19 sequence is available under GENBANK accession number AAA69966. In particular embodiments, the CAR of this invention includes a human CD33 cytoplasmic domain (amino acids 52-164; SEQ ID NO: 11), human CD28 hinge-transmembrane-cytoy plasmic domains (amino acids 135-220; SEQ ID NO: 12), and optionally a portion of CD19 (amino acids 1-327; SEQ ID NO: 13).

Other synthetic molecules include therapeutic agents such as cytotoxic, cytostatic, or anti-angiogenic agents and radioisotopes. A cytotoxic agent can be a plant, fungal, or bacterial molecule (e.g., a protein toxin). A therapeutic agent can be a maytansinoid (e.g., maytansinol or DM1 maytansinoid), a taxane, or a calicheamicin. Therapeutic agents include vincristine and prednisone. A therapeutic agent can be an antimetabolite (e.g., an antifolate such as methotrexate, a fluoropyrimidine such as 5-fluorouracil, cytosine arabinoside, or an analogue of purine or adenosine); an intercalating agent (for example, an anthracycline such as doxorubicin, daunomycin, epirubicin, idarubicin, mitomycin-C, dactinomycin, or mithramycin); a platinum derivative (e.g., cisplatin or carboplatin); an alkylating agent (e.g., nitrogen mustard, melphalan, chlorambucil, busulphan, cyclophosphamide, ifosfamide nitrosoureas or thiotepa); an antimitotic agent (e.g., a vinca alkaloid like vincristine or taxoid such as paclitaxel or docetaxel); a topoisomerase inhibitor (for example, etoposide and teniposide, amsacrine, topotecan); a cell cycle inhibitor (for example, a flavopyridol); or a microbtubule agent (e.g., an epothilone, discodermolide analog, or eleutherobin analog). A therapeutic agent can be a proteosome inhibitor or a topoisomerase inhibitor such as bortezomib, amsacrine, etoposide, etoposide phosphate, teniposide, or doxorubicin. Therapeutic radioisotopes include yttrium (^{90Y}), lutetium (¹⁷⁷Lu), actinium (²²⁵Ac), praseodymium, astatine (²¹¹At) rhenium (¹⁸⁶Re), bismuth (²¹²Bi or ²¹³Bi), and rhodium (¹⁸⁸Rh). Antiangiogenic agents include linomide, bevacuzimab, angiostatin, and razoxane. The synthetic molecule can be another antibody such as rituximab or bevacuzimab.

A synthetic molecule can also be a label. Labels can be useful in diagnostic applications and can include, for example, contrast agents. A contrast agent can be a radioisotope label such as iodine (¹³¹I or ¹²⁵I) indium (¹¹¹In), technetium (⁹⁹Tc), phosphorus (³²P), carbon (¹⁴C), tritium (³H), other radioisotope (e.g., a radioactive ion) or a therapeutic radioisotope listed above. Additionally, contrast agents can include radiopaque materials, magnetic resonance imaging (MRI) agents, ultrasound imaging agents, and any other contrast agents suitable for detection by a device that images an animal body. A synthetic molecule can also be a fluorescent label, a biologically active enzyme label, a luminescent label, or a chromophore label.

Moreover, a synthetic molecule can also be a magnetic nanoparticle, a controlled release polymer nanoparticle or lipid composition. Magnetic nanoparticles include, but are not limited to iron (e.g., Fe₃O₄ or Fe₂O₄), cobalt, zinc, cadmium, nickel, gadolinium, chromium, copper, manganese, terbium, europium, gold, silver, platinum, or alloys thereof. Controlled release polymer nanoparticles can be produced using conventional methods from biodegradable or nonbiodegradable polymers, e.g., poly (lactic acid), derivatives of poly (lactic acid), PEGylated poly(lactic acid), poly (lactic-co-glycolic acid), derivatives of poly (lactic-co-glycolic acid), PEGylated poly (lactic-co-glycolic acid), a polyanhydride, poly(ortho esters), derivatives of poly(ortho esters), PEGylated poly(ortho esters), poly (caprolactone), derivatives of poly (caprolactone), PEGylated poly(caprolactone), poly(acrylic acid), derivatives of poly (acrylic acid), poly(urethane), derivatives of poly (urethane), or combinations thereof). Similarly, lipid composition (e.g., liposomes, solid lipid nanoparticles and the like) can be produced using conventional methods and conjugated to an antibody of this invention.

In some embodiments, the antibody can also have specificity for one or more antigens in addition to B7-H6. For example, the antibody of the invention can be engineered (e.g., as a bivalent diabody or a conjugated Fab dimer or trimer) to have specificity for B7-H6 and another tumor antigen, e.g., an antigen associated with a lymphoma, leukemia, melanoma, or sarcoma disclosed herein. Alternatively, the antibody can be engineered to have specificity for B7-H6 and an antigen that promotes activation or targeting of other cells, such as cytotoxic effector cells or T cells. Accordingly, the invention also includes BiTES (bi-specific T-cell engagers) and DARTS(dual affinity retargeting reagents).

As is known in the art, a BiTE refers to a single polypeptide chain molecule that having two antigen binding domains, one of which binds to a T-cell antigen (e.g., CD3) and the second of which binds to an antigen present on the surface of a target cell (WO 05/061547; Baeuerle, et al. (2008) Drugs of the Future 33:137-147; Bargou, et al. (2008) Science 321:974-977). BiTE antibodies have been constructed to various target antigens including CD19, EpCAM, Her2/neu, EGFR, CD66e (or CEA, CEACAM5), CD33, EphA2, and MCSP (or HMW-MAA) (Baeuerle, et al. (2009) Curr. Opin. Mol. Ther. 11:22-30). Key hallmarks of BiTE antibodies that, in their combination, distinguish them from other bispecific antibody constructs, include a high potency of redirected lysis with EC50 values ranging from 0.1 to 50 pmol/L (2-1,000 pg/mL) (Baeuerle, et al. (2009) supra); strict target cell-dependent activation of T cells (Brischwein, et al. (2007) J. Immunother. 30:798-807); and support of serial lysis by activated T cells, i.e., activity at low E:T ratios. BiTE antibodies are typically produced as recombinant, glycosylated proteins secreted by higher eukaryotic cell lines. Accordingly, in another embodiment of this invention, an anti-B7-H6 antibody fragment (e.g., a scFv) is a component of a BiTE. In particular embodiments, the BiTE of this invention is composed of an anti-B7-H6 antibody fragment and an anti-CD3 antibody fragment fused together by a linker, the(G4S)3 e.g., linker. In specific embodiments, the anti-CD3 antibody fragment includes a heavy chain variable region having a CDR1 sequence of Ser-Gly-Tyr-Thr-Phe-Thr-Arg-Tyr-Thr-Met-His (SEQ ID NO:15), CDR2 sequence of Tyr-Ile-Asn-Pro-Ser-Arg-Gly-Tyr-Thr-Asn-Tyr-Asn-Gln-Lys-Phe-Lys-Asp (SEQ ID NO: 16), and CDR3 sequence of Tyr-Tyr-Asp-Asp-His-Tyr-Cys-Leu (SEQ ID NO: 17); and a light chain variable region having a CDR1 sequence of Ser-Ala-Ser-Ser-Ser-Val-Ser-Tyr-Met-Asn (SEQ ID NO: 18), CDR2 sequence of Asp-Thr-Ser-Lys-Leu-Ala-Ser (SEQ ID NO: 19) and CDR3 sequence of Gln-Gln-Trp-Ser-Ser-Asn-Pro-Phe (SEQ ID NO: 20). See U.S. Pat. No. 5,929,212.

A DART refers to an immunoglobulin molecule that includes at least two polypeptide chains that associate (especially through a covalent interaction) to form at least two epitope binding sites, which may recognize the same or different epitopes. Each of the polypeptide chains of a DART include an immunoglobulin light chain variable region and an immunoglobulin heavy chain variable region, but these regions do not interact to form an epitope binding site. Rather, the immunoglobulin heavy chain variable region of one (e.g., the first) of the DART polypeptide chains interacts with the immunoglobulin light chain variable region of a different (e.g., the second) DART polypeptide chain to form an epitope binding site. Similarly, the immunoglobulin light chain variable region of one (e.g., the first) of the DART polypeptide chains interacts with the immunoglobulin heavy chain variable region of a different (e.g., the second) DART polypeptide chain to form an epitope binding site. DARTs may be monospecific, bispecific, trispecific, etc., thus being able to simultaneously bind one, two, three or more different epitopes (which may be of the same or of different antigens). DARTs may additionally be monovalent, bivalent, trivalent, tetravalent, pentavalent, hexavelent, thus etc., being able to simultaneously bind one, two, three, four, five, six or more molecules. These two attributes of DARTs (i.e., degree of specificity and valency may be combined, for example to produce bispecific antibodies (i.e., capable of binding two epitopes) that are tetravalent (i.e., capable of binding four sets of epitopes), etc. The construction of DART molecules is disclosed in WO 2006/113665, WO 2008/157379, and WO 2010/080538. Accordingly, in another embodiment of this invention, an anti-B7-H6 antibody fragment is included in a DART.

The invention further provides eukaryotic or non-eukaryotic cells that have been recombinantly engineered to produce an antibody of the invention. The eukaryotic or non-eukaryotic cells can be used as an expression system to produce the antibody of the invention. In another embodiment, the invention provides B7-H6 targeted immune cells that are engineered to recombinantly express a B7-H6 specific antibody of the invention. For example, the invention provides a T-cell engineered to express an antibody of the invention (e.g., an scFv, scFv-Fc, (scFv)₂), which is linked to a synthetic molecule with the following domains: a spacer or hinge region (e.g., a CD28 or IgG hinge), a transmembrane region (e.g., a transmembrane canonical domain), and an intracellular T-cell receptor (TCR) signaling domain, thereby forming a CAR. Intracellular TCR signaling domains that can be included in a CAR include, but are not limited to, CD3zeta, FcR-gamma and Syk-PTK signaling domains as well as the CD28, 4-1BB, and CD134 co-signaling domains. Methods for constructing T-cells expressing a CAR are known in the art. See, e.g., Marcu-Malina, et al. (2009) Exp. Opin. Biol. Ther. 9:579-91.

The invention provides a method of inhibiting cells that express B7-H6 (B7-H6 cells) by contacting the cells with an antibody, antibody fragment or fusion protein (e.g., BiTE) of the invention. The antibody can be a naked (unconjugated) antibody or an antibody conjugated to a synthetic molecule, e.g., a cytotoxic, cytostatic, or anti-angiogenic agent or a radioisotope. The method can be used to inhibit B7-H6 cells in vitro or in a subject (i.e., in vivo). The contacted B7-H6 cells can be in, for example, a cell culture or animal model of a disorder associated with aberrant expression or levels of B7-H6. The method is useful, for example, to measure and/or rank (relative to another antibody) the antibody's inhibitory activity for a specific B7-H6 cell type. Inhibiting B7-H6 cells can include blocking or reducing the activity or growth of B7-H6-positive cells (i.e., cells that express B7-H6). Inhibiting can also include the killing of B7-H6-positive cells. Cytotoxicity of an antibody, antibody fragment or fusion protein (e.g., BiTE) of the invention can be assessed using a lactate any conventional assay including, e.g., dehydrogenase cytotoxicity assay such as the CYTOTOX 96 non-radioactive cytotoxicity assay commercially available from PROMEGA.

The invention also provides a method of treating a subject that has, is suspected to have, or is at risk for a disorder associated with aberrant levels of B7-H6. As used in the context of the present invention, the term “aberrant” is intended to include increased or decreased B7-H6 expression as compared to expression of B7-H6 in normal or healthy cells. In this respect, wherein a normal cell does not express B7-H6 and a diseased cell B7-H6, the diseased cell exhibits aberrant expression of B7-H6. Generally, the method of treatment includes administering a therapeutically effective amount of an isolated antibody, antibody fragment or fusion protein of the invention to the subject. The antibody can be any anti-B7-H6 antibody described herein. Thus, the antibody can be chimeric, humanized, synthetic, F(ab)₂, Fv, scFv, F(ab′)₂, F(ab), VL, VH, dsFv, Fv, or (scFv)₂. In some embodiments, the method includes administering an IgG, an scFv, a dsFv, a F(ab′)₂, a diabody, a bivalent antibody, a CAR, a BITE or a DART. In other embodiments, the administered antibody can be conjugated to a synthetic molecule described above, e.g., a cytotoxic, cytostatic, or anti-angiogenic agent or a therapeutic radioisotope. An exemplary cytotoxic agent is Pseudomonas exotoxin A (PE38). Disorders that can be treated include, for example, lymphomas, leukemia, melanomas, and sarcomas. Particular disorders associated with elevated B7-H6 that can be treated include myeloid leukemia (e.g., acute myeloid leukemia), acute nonlymphocytic leukemia, T-cell acute lymphoblastic leukemia, T- or B-cell lymphoma, cervical cancer, gastric sarcoma, breast cancer, pancreatic cancer, melanoma, or prostate cancer.

The invention also provides a method of treating a subject that has, is suspected to have, or is at risk for a disorder associated with elevated levels of B7-H6 by adoptive transfer of the recombinant host cells, e.g., T-cells described herein, which express an antibody of the invention as a CAR or BiTE that selectively binds B7-H6. Recombinant technology can be used to introduce CAR- or BiTE-encoding genetic material into any suitable T-cells, e.g., effector memory T-cells from the subject to be treated. The T-cells carrying the genetic material can be expanded (e.g., in the presence of cytokines). The recombinant T-cells are transferred, typically by infusion, to the patient. The transferred T-cells of the invention can then mount an immune response against B7-H6 expressing cells in the subject. The adoptive transfer method can be used, for example, to treat subjects that have or are suspected to have myeloid leukemia, acute nonlymphocytic leukemia, T-cell acute lymphoblastic leukemia, T- or B-cell lymphoma, cervical cancer, gastric sarcoma (e.g., colon cancer), breast cancer, pancreatic cancer, melanoma, or prostate cancer.

In some embodiments, the foregoing methods of treatment can further include co-administering a second therapeutic agent for the disorder associated with elevated B7-H6. For example, when the disorder to be treated involves a B7-H6-expressing cancer, the method can further include co-administration of a cytotoxic, cystostatic, or anti-angiogenic agent suitable for treating the cancer. If the cancer is a B-cell lymphoma, the method can further include, for example, co-administration of rituximab, alemtuzumab, or a CHOP chemotherapeutic regimen.

The terms “treat,” “treating,” “treatment,” and “therapeutically effective” used herein do not necessarily imply 100% or complete treatment. Rather, there are varying degrees of treatment of which one of ordinary skill in the art recognizes as having a potential benefit or therapeutic effect. In this respect, the inventive method can provide any amount of any level of treatment. Furthermore, the treatment provided by the inventive method can include the treatment of one or more conditions or symptoms of the disease being treated.

For use in treatment, the invention also provides a pharmaceutical composition containing an antibody as described herein and a pharmaceutically acceptable carrier. Pharmaceutical compositions can be prepared from any of the antibodies described herein. An exemplary composition includes a chimeric antibody having SEQ ID NO: 3 (heavy chain) and/or SEQ ID NO: 4 (light chain). Another exemplary composition includes a humanized antibody having one, two, three, four, five, or six CDRs selected from the group of SEQ ID NO: 5, SEQ ID NO: 6, SEQ ID NO: 7, SEQ ID NO: 8, SEQ ID NO: 9, and SEQ ID NO: 10. Still another exemplary pharmaceutical composition includes anti-B7-h6 scFv fused to anti-CD3e scFv via a flexible linker (i.e., a BiTE). Yet another exemplary pharmaceutical composition includes anti-B7-h6 SCFv fused to the hinge, transmembrane and intracellular domains of CD28 and the intracellular domain of CD3zeta (i.e., a CAR).

The composition of the invention can include a carrier for the antibody, desirably a pharmaceutically acceptable carrier. The pharmaceutically acceptable carrier can be any suitable pharmaceutically acceptable carrier. The term “pharmaceutically acceptable carrier,” as used herein, means one or more compatible solid or liquid fillers, diluents, other excipients, or encapsulating substances, which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the application. The pharmaceutically acceptable carrier can be co-mingled with one or more of the active components, e.g., a hybrid molecule, and with each other, when more than one pharmaceutically acceptable carrier is present in the composition in a manner so as not to substantially impair the desired pharmaceutical efficacy. “Pharmaceutically acceptable” materials typically are capable of administration to a patient without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

The pharmaceutical composition can contain suitable buffering agents, including, for example, acetic acid in a salt, citric acid in a salt, boric acid in a salt, and phosphoric acid in a salt. The pharmaceutical compositions also optionally can contain suitable preservatives, such as benzalkonium chloride, chlorobutanol, parabens, and thimerosal.

The pharmaceutical composition can be presented in unit dosage form and, can be prepared by any suitable method, many of which are well-known in the pharmaceutical arts. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

A composition suitable for parenteral administration conveniently includes a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland, fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, Mack Publishing Co., Easton, PA.

The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: erosional systems in which the active composition is contained in a form within a matrix such as those described in U.S. Pat. Nos. 4,452,775, 4,667,014, 4,748,034, and 5,239,660; and diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

The term “subject” is used herein, for example, in connection with therapeutic and diagnostic methods, to refer to human or animal subjects. Animal subjects include, but are not limited to, animal models, such as, mammalian models of conditions or disorders associated with elevated B7-H6 expression such as myeloid leukemia, acute nonlymphocytic leukemia, T-cell acute lymphoblastic leukemia, T- or B-cell lymphoma, cervical cancer, gastric sarcoma (e.g., colon cancer), breast cancer, pancreatic cancer, melanoma, or prostate cancer.

In another embodiment, the invention provides use of the antibodies of the invention to detect in a test sample an altered amount of B7-H6 (e.g., cell surface B7-H6), for example, relative to a control. A test sample can be from a cell culture or from a test subject, e.g., a plasma or a tissue sample from a subject that has, is suspected to have, or is at risk for a disease or condition associated with aberrant expression of B7-H6 in a subject. A control amount desirably corresponds to the B7-H6 amount detected using the same antibody in a corresponding sample(s) from one or more control cultures or subjects. Methods of using the antibody of the invention to determine B7-H6 amounts can include any immunoassay such as immuno-(western) blot, enzyme-linked immunosorbent assay (ELISA), and flow cytometry, e.g., fluorescence-activated cell sorting (FACS) analysis.

Additionally, B7-H6 detection can be used to monitor the progress of a disorder associated with B7-H6 expression. Amounts of B7-H6 that are significantly elevated or decreased relative to control indicate that the subject's disorder is deteriorating or improving, respectively.

The foregoing screens can be used to identify the presence or to monitor the progress of disorders including, for example, myeloid leukemia, acute nonlymphocytic leukemia, T-cell acute lymphoblastic leukemia, T- or B-cell lymphoma, cervical cancer, gastric sarcoma (e.g., colon cancer), breast cancer, pancreatic cancer, melanoma, or prostate cancer.

The invention also provides kits suitable for carrying out the methods of the invention. Typically, a kit includes two or more components required for performing a therapeutic or detection method of the invention. Kit components include, but are not limited to, one or more antibody of the invention, appropriate reagents, and/or equipment.

A kit can include an antibody of the invention and an immunoassay buffer suitable for detecting B7-H6 (e.g., by ELISA or FACS). The kit may also contain one or more microliter plates, standards, assay diluents, wash buffers, adhesive plate covers, and/or instructions for carrying out a method of the invention using the kit. The kit can include an antibody of the invention bound to a substrate (e.g., a multi-well plate or a chip), which is suitably packaged and useful to detect B7-H6. In some embodiments, the kit includes an antibody of the invention that is conjugated to a label, such as, a fluorescent label, a biologically active enzyme label, a luminescent label, or a chromophore label. The kit can further include reagents for visualizing the conjugated antibody, e.g., a substrate for the enzyme. In some embodiments, the kit includes an antibody of the invention that is conjugated to a contrast agent and, optionally, one or more reagents or pieces of equipment useful for imaging the antibody in a subject.

Generally the antibody of the invention in a kit is suitably packaged, e.g., in a vial, pouch, ampoule, and/or any container appropriate for a therapeutic or detection method. Kit components can be provided as concentrates (including lyophilized compositions), which may be further diluted prior to use or they can be provided at the concentration of use. When the antibody of the invention for use in vivo, single dosages may be provided in sterilized containers having the desired amount and concentration of agents.

The invention is described in greater detail by the following non-limiting examples.

### Example 1: Materials and Methods

Construction of an Anti-B7H6 Chimeric Antigen Receptor. Anti-B7H6 CAR was constructed by fusing single change variable fragment (scFv) of anti-B7H6 hybridoma clone 47.39 to human CD28 hinge-transmembrane-cytoplasmic domains (residues 135-220) and CD3 zeta cytoplasmic domain (residues 52-164). The construction of anti-B7H6 scFv was done by fusing variable region of heavy chain (V_{H}) and light chains (V_{L}) with a 15 amino acid glycine (G)-serine (S) linker ((G₄S)₃ linker; 3 repeats of GGGGS (SEQ ID NO: 14)). Anti-B7H6 construct was then cloned into a retroviral vector pFB-neo (Stratagene, Palo Alto, CA). Anti-B7H6 CAR-T2A-tCD19 construct was made by fusing anti-B7H6 CAR to T2A sequence, followed by truncated human CD19 (residues 1-327). All PCRs were done by using high-fidelity DNA polymerase Phusion (New England BioLabs, Ipswich, MA).

Retroviral Transduction. Transduction of murine primary T cells was conducted using ecotropic viruses collected from vector-transduced GP+E86 cells, whereas dualtropic retroviral viruses generated from vector-transduced PT67 cells were used to infect human primary T cells. Primary T cells from spleens of B6 mice were infected 18-24 hours after concanavalin A (ConA, 1 μg/ml) stimulation. Two days after infection, transduced primary T cells (0.5˜1×10⁶/ml) were selected in RPMI-10 media containing G418 (1 mg/ml) plus 25 U/ml rHuIL-2 for additional 3 days. Viable cells were isolated using HISTOPAQUE-1083 (Sigma, St. Louise, MO), washed extensively, and expanded for 2 days without G418 before functional analyses or intravenous injection. Primary human T cells from cell clones were activated with anti-CD3 mAb OKT3 (40 ng/ml; eBioscience) plus IL-2 (50 U/mL) for 3 days before transduction. G418 selection and T cell expansion were done following similar procedures for culturing mouse T cells.

Construction of an Anti-B7H6 BiTE. Anti-B7H6 scFv was fused via a flexible linker to an anti-OKT3 scFv (i.e, anti-CD3 scFv; Arakawa, et al. (1996) J. Biochem. 120:657-62; U.S. Pat. No. 5,929,212, incorporated by reference herein in its entirety) (FIG. 6A). The resulting human BiTE protein(huBiTE) was expressed in retroviral vector-stably transfected PT67 packaging cells. The resulting cells were then cultured in serum-free media. Supernatants were collected and tumor cell cytotoxicity of the anti-B7H6 BiTE was assessed using a conventional lactate dehydrogenase assay (FIG. 6B). This analysis showed that the anti-B7H6 BiTE could specifically lyse B7H6⁺ tumor cells.

To determine whether anti-B7H6 BiTE could engage both T cells and tumor cells and lead to T cell activation, OKT3-activated T cells were co-cultured with tumor cells (RMA, RMA-B7H6, and K562) with or without anti-B7H6 BiTE for 24 hours. Amounts of IFN-γ in supernatants were analyzed with ELISA. This analysis indicated that the anti-B7H6 BiTE induced IFN-γ secretion into the medium of T cells co-cultured with tumor cells expressing B7H6, i.e., RMA-B7H6, and K562 (FIGS. 7A and 7B). Similarly, a mouse anti-B7H6 BiTE (MuBiTEl) was added to a co-culture of ConA-activated splenocytes and tumor cells and shown to trigger robust IFN-γ secretion in the T cell and tumor cell co-culture system (FIG. 7C). Moreover, like the huBiTE, muBiTE specifically activated T cells to kill B7H6+ tumor cells (FIGS. 8A and 8B).

What is claimed is:

1. An isolated antibody, or antigen binding fragment of the antibody, which specifically binds to B7 homolog 6 (B7-H6) comprising:
(a) a heavy chain variable region comprising,
(i) a CDR1 of SEQ ID NO: 5,
(ii) a CDR2 of SEQ ID NO: 6, and
(iii) a CDR3 of SEQ ID NO: 7; and
(b) a light chain variable region comprising,
(i) a CDR1 of SEQ ID NO: 8,
(ii) a CDR2 of SEQ ID NO: 9, and
(iii) a CDR3 of SEQ ID NO: 10.

2. The antibody of claim 1, wherein
(i) the antibody comprises a heavy chain variable domain of SEQ ID NO: 3 and/or comprises a light chain variable domain of SEQ ID NO: 4;
(ii) the antibody is humanized;
(iii) the antibody is conjugated to a synthetic molecule;
(iv) the antibody is comprised in a chimeric antigen receptor (CAR) which CAR comprises a transmembrane region and an intracellular T-cell receptor signaling domain, optionally wherein the transmembrane region and intracellular T-cell receptor signaling domain are from CD3 zeta, and further optionally wherein the CAR further comprises the intracellular domain of a costimulatory protein receptor; or
(v) any combination og (i) to (iv).

3. (canceled)

4. (canceled)

5. (canceled)

6. (canceled)

7. (canceled)

8. (canceled)

9. A recombinant T cell comprising the antibody of claim 1.

10. The antibody of claim 2, wherein the antibody is a bi-specific T-cell engager and the synthetic molecule comprises an antigen binding domain which binds to a T-cell antigen, optionally CD3.

11. (canceled)

12. The antibody of claim 10, wherein the bi-specific T-cell engager comprises an antibody fragment that specifically binds CD3 which comprises:
(a) a heavy chain variable region comprising,
(i) a CDR1 of SEQ ID NO: 15,
(ii) a CDR2 of SEQ ID NO: 16, and
(iii) a CDR3 of SEQ ID NO: 17; and
(b) a light chain variable region comprising,
(i) a CDR1 of SEQ ID NO: 18,
(ii) a CDR2 of SEQ ID NO: 19, and
(iii) a CDR3 of SEQ ID NO: 20.

13. The antibody of claim 2, wherein the synthetic molecule is a label, a cytotoxic agent or a therapeutic radioisotope.

14. (canceled)

15. A pharmaceutical composition comprising the antibody or antigen binding fragment of claim 1 and a pharmaceutically acceptable carrier.

16. A kit comprising the antibody or antigen binding fragment of claim 1.

17. The kit of claim 16, wherein the antibody or antigen binding fragment is conjugated to a label.

18. A chimeric antigen receptor comprising
(a) an antigen binding fragment of an antibody that specifically binds to B7 homolog 6,
(b) a transmembrane region, and
(c) an intracellular T-cell receptor signaling domain.

19. The chimeric antigen receptor of claim 18, wherein the transmembrane region and intracellular T-cell receptor signaling domain are from CD3 zeta.

20. The chimeric antigen receptor of claim 18, further comprising a transmembrane domain or an intracellular signaling domain of a costimulatory protein receptor.

21. The chimeric antigen receptor of claim 18, further comprising a hinge region or spacer region of to create flexibility or increased intercellular spacing.

22. A recombinant T cell comprising the chimeric antigen receptor of claim 18.

23. A bi-specific T-cell engager comprising
(a) an antigen binding fragment of an antibody that specifically binds to B7 homolog 6, and
(b) an antigen binding domain which binds to a T-cell antigen, optionally CD3.

24. (canceled)

25. The bi-specific T-cell engager of claim 22, wherein the antibody fragment that specifically binds CD3 comprises:
(a) a heavy chain variable region comprising,
(i) a CDR1 of SEQ ID NO: 15,
(ii) a CDR2 of SEQ ID NO: 16, and
(iii) a CDR3 of SEQ ID NO: 17; and
(b) a light chain variable region comprising,
(i) a CDR1 of SEQ ID NO: 18,
(ii) a CDR2 of SEQ ID NO: 19, and
(iii) a CDR3 of SEQ ID NO: 20.

26. A method for killing or inhibiting the growth of cells expressing B7 homolog 6 in a subject comprising administering an effective amount of an antibody of claim 1 or a CAR or a bi-specific T-cell engager containing to a subject in need thereof, thereby killing or inhibiting the growth of cells expressing B7 homolog 6 in the subject.

27. A method of treating a disease or condition associated with expression of B7 homolog 6 in a subject comprising administering an effective amount of an antibody 1 or a CAR or a bi-specific T-cell engager containing to a subject in need thereof, thereby treating a disease or condition associated with elevated expression of B7 homolog 6 in the subject.

28. The method of claim 27, wherein the disease or condition is myeloid leukemia, acute nonlymphocytic leukemia, T-cell acute lymphoblastic leukemia, T-cell lymphoma, B-cell lymphoma, cervical cancer, gastric sarcoma, breast cancer, pancreatic cancer, melanoma, or prostate cancer.

29. The method of claim 27, further comprising co-administering a pharmaceutical composition comprising a second therapeutic agent.


SEQUENCE LISTING (20 sequences)

SEQ ID NO: 1 - DNA, 1418 bp, synthetic construct
  misc_feature 1..1418: note = Synthetic heavy chain sequence
  source 1..1418: mol_type = other DNA
atggaatggacctgggtctttctcttcctcctgtcagtaactgcaggtgtccactcccaggttcagctgcagcagtctggagctgagctgatgaagcctggggcctcagtgaagctttcctgcaaggctactggctacacattcactggctactggatagagtggataaagcagaggcctggacatggccttgagtggattggagagattttacctggaactggtagtactaactacaatgagaagttcaagggcaaggccacattcactgcagatacatcctccaacacagcctacatgcaactcagcagcctgacaactgaggactctgccatctattactgtgcaatcccggggcctatggactactggggtcaaggaacctcagtcaccgtctcctcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggactccgggtaaatgagcggccgc

SEQ ID NO: 2 - DNA, 719 bp, synthetic construct
  misc_feature 1..719: note = Synthetic light chain sequence
  source 1..719: mol_type = other DNA
atggacatgaggacccctgctcagtttcttggaatcttgttgctctggtttccaggtatcaaatgtgacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaggcggccgc

SEQ ID NO: 3 - protein, 121 aa, synthetic construct
  REGION 1..121: note = Synthetic heavy chain sequence
QVQLQQSGAELMKPGASVKLSCKATGYTFTGYWIEWIKQRPGHGLEWCDRIGEILPGTGSTNYNEKFKGKATFTADTSSNTAYMQLSSLTTEDSAIYCDRYCAIPGPMDYWGQGTSVTVSS

SEQ ID NO: 4 - protein, 116 aa, synthetic construct
  REGION 1..116: note = Synthetic light chain sequence
DIKMTQSPSSMYASLGERVTITCKASQDINSYLSWFQQKPGKSPKTLCDRIYRANRLVDGVPSRFSGSGSGQDYSLTISSLEYEDMGIYYCLQYDEFCDRCDRPYTFGGGTKLEIK

SEQ ID NO: 5 - protein, 8 aa, synthetic construct
  REGION 1..8: note = Synthetic heavy chain variable region CDR1
GYTFTGYW

SEQ ID NO: 6 - protein, 8 aa, synthetic construct
  REGION 1..8: note = Synthetic heavy chain variable region CDR2
ILPGTGST

SEQ ID NO: 7 - protein, 8 aa, synthetic construct
  REGION 1..8: note = Synthetic heavy chain variable region CDR3
AIPGPMDY

SEQ ID NO: 8 - protein, 6 aa, synthetic construct
  REGION 1..6: note = Synthetic light chain variable region CDR1
QDINSY

SEQ ID NO: 9 - skipped

SEQ ID NO: 10 - protein, 9 aa, synthetic construct
  REGION 1..9: note = Synthetic light chain variable region CDR3
LQYDEFPYT

SEQ ID NO: 11 - protein, 113 aa, synthetic construct
  REGION 1..113: note = Synthetic CD3 zeta cytoplasmic domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 86 aa, synthetic construct
  REGION 1..86: note = Synthetic CD28 hinge-transmembrane-cytoplasmic domains
VKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 13 - protein, 327 aa, synthetic construct
  REGION 1..327: note = Synthetic CD19
MPPPRLLFFLLFLTPMEVRPEEPLVVKVEEGDNAVLQCLKGTSDGPTQQLTWSRESPLKPFLKLSLGLPGLGIHMRPLASWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 14 - protein, 15 aa, synthetic construct
  REGION 1..15: note = Synthetic linker peptide
GGGGSGGGGSGGGGS

SEQ ID NO: 15 - protein, 11 aa, synthetic construct
  REGION 1..11: note = Synthetic CD3 HCVR CDR1
SGYTFTRYTMH

SEQ ID NO: 16 - protein, 17 aa, synthetic construct
  REGION 1..17: note = Synthetic CD3 HCVR CDR2
YINPSRGYTNYNQKFKD

SEQ ID NO: 17 - protein, 8 aa, synthetic construct
  REGION 1..8: note = Synthetic CD3 HCVR CDR3
YYDDHYCL

SEQ ID NO: 18 - protein, 10 aa, synthetic construct
  REGION 1..10: note = Synthetic CD3 LCVR CDR1
SASSSVSYMN

SEQ ID NO: 19 - protein, 7 aa, synthetic construct
  REGION 1..7: note = Synthetic CD3 LCVR CDR2
DTSKLAS

SEQ ID NO: 20 - protein, 39 aa, synthetic construct
  REGION 1..39: note = Synthetic CD3 LCVR CDR3
GLNGLNTRPSERSERASNPROPHESEQUENCELISTING